(19) United States
(12) Patent Application Publication
(10) Pub. No.: US 2009/0286674 A1
(43) Pub. Date: Nov. 19, 2009

(54) ACTIVATION OF SURFACES THROUGH GAS PHASE REACTIONS

(75) Inventors: Helmer Fjellvag, Oslo (NO); Ola Nilsen, Oslo (NO)

Correspondence Address: VOLPE AND KOENIG, P.C., UNITED PLAZA, SUITE 1600, 30 SOUTH 17TH STREET, PHILADELPHIA, PA, 19103, US

(73) Assignee: UNIVERSITETET I OSLO, Oslo (NO)

(21) Appl. No.: 12/305,565

(22) PCT Filed: Jun. 19, 2007
(86) PCT No.: PCT/NO2007/000217

§ 371 Date: Dec. 18, 2008

(30) Foreign Application Priority Data
Jun. 19, 2006 (NO) 20062871

(51) Int. Cl. B01J 31/02; C07C 215/24; C23C 16/00; C23C 16/06

(52) U.S. Cl. 502/167; 564/507; 427/255.28; 427/250; 427/255.38; 427/255.394; 427/255.39

(57) ABSTRACT

A process for the preparation of active surfaces terminated by a desired form of organic, organic-inorganic, or inorganic nature comprising growing with a gas phase deposition technique preferable the ALCVD (atomic layer chemical vapour deposition) technique. As an example, trimethylaluminium (TMA), hydroquinone (Hq) and phloroglucinol (Phl) have been used as precursors to fabricate surfaces that are terminated by hydroxyl groups attached to aromates. Further types of active surfaces are described. These surfaces can be used to produce surfaces: suitable for adhesion through the use of glue or other adhesive, providing receptors for biological molecules, making the surfaces biocompatible, of catalytically active materials, where upon subsequent types of chemical reactions can take place, with different degrees of wetting properties.

The present invention relates to a process for preparation of surfaces that are terminated with a specific type of molecules, thus producing an active surface, the active surface, substrates comprising the active surface, the use of the active surface, and a device comprising the active surface.

ALCVD (=atomic layer chemical vapour deposition, also known as ALE=atomic layer epitaxy, and ALD=atomic layer deposition) is a thin film technique that utilizes only surface reactions, and is described in prior art, see e.g. M. Ritala, M. Leskelä, in: H. S. Nalwa (Ed.), Handbook of Thin Film Materials, vol. I, Academic Press, San Diego, Calif., 2001, p. 103.

A thin film is produced by the ALCVD technique by using different types of precursors. The precursors are pulsed sequentially into the reaction chamber where it reacts with all surfaces present; each pulse is followed by a purging time with an inert gas. In this way gas phase reactions are eliminated and film is constructed by precursor units in the order that they are pulsed. This technique makes it possible to change building units at the resolution of one monolayer, and therefore enables production of artificial structures of hybrid films with different types of organic and inorganic building units.

It has now surprisingly been found that this technique can be utilized to construct active surfaces comprising a desired type of molecule, thereby providing active surfaces with desired properties and different types of chemical nature. The molecules terminating the surface are produced with the ALCVD method, by choosing the last pulse to be of the type of precursor that results in the active surface that is desired.

An active surface in this context is a surface that is terminated by a type of chemical sites that are suitable for a certain type of applications. To activate a surface in this context is to produce such active sites on a native surface without these sites. The active sites may be any type of functional groups known in organic and inorganic chemistry, and combinations thereof. Examples of functional groups in organic chemistry are: hydroxides, carboxylic acids, ketones, amines, amides, halogenides, alkyls, aromates, alkylenes, alkylyns, thioles, phosphates, nitroxyls, sulphates and amino acids. Examples of functional groups in inorganic chemistry are metal oxides, metal sulphides, metal nitrides, metal borides, metal carbonyls, metal phosphates and metals.

Active surfaces can at present be produced by many different means such as wet types of impregnation, dip coating, self assembled monolayers, etc. The surprisingly additional effect that the ALCVD technique brings into this field is the possibility to use alternating highly reactive types of precursors in order to produce an active surface. For instance it will be difficult, if not impossible, to produce a surface terminating in an organic type of molecule on an aluminium surface or in fact aluminium oxide surface without using additional metal elements such as silicon. This is a known phenomenon when it comes to producing glue that sticks on aluminium surfaces by coating them with an organosilane, and is for instance described in “Understanding the relationship between silane application conditions, bond durability and locus of failure” M.-L. Abela, R. D. Allingtonb, R. P. Digbyc, N. Porrittd, S. J. Shawb, and J. F. Wattsa, International Journal of Adhesion and Adhesives, February-April 2006, Pages 2-15; E. P. Plueddemann, “Silane coupling agents”, Plenum Press, New York (1982).

Using the ALCVD technique, it will for instance be possible to produce surfaces terminated by organic type of molecules by the aid of the highly reactive trimethyl aluminium precursor. By introducing one pulse of this precursor, all surfaces present will be terminated by an aluminium-methyl complex. This is highly reactive and will react further with organic molecules with suitable types of sites. The procedure can be explained through an example:

1) Saturation of surface with hydroxyl groups. Normally all surfaces are terminated by hydroxyl groups, but in order to improve the saturation density, or just to be on the safe side, it is beneficial to begin the deposition with one pulse of water, and thus saturate the surfaces with hydroxyl groups.
2) Saturation of surface with trimethyl aluminium. By pulsing trimethyl aluminium, it will react with all available hydroxyl groups and form a methyl aluminium terminated surface.
3) Binding of the organic molecule to the aluminium methyl terminated surface. By choosing a type of precursor that has at least one type of functional group suitable for reaction with the methyl aluminium surface, one will be able to saturate the surface with this molecule. One example would be to use carboxylic acids as a functional group for reaction with the methyl aluminium surface. The organic molecule would then be HOOC—R, where the -R designates the type of organic molecule one requires the surface to be terminated with. Other functional groups are alcohols, amines, etc. However, experience has shown that for trimethyl aluminium, carboxylic acids give stable bonds to the surface and thus is very suited, and will result in the following top layer structure

|—O—Al—OOC—R

The use of ALCVD technique to produce such active surfaces is not yet described in the public available literature.

Active surfaces may be used for many different types of purposes, some being:
To produce a surface suitable for adhesion through the use of glue or other means.
To produce a surface providing receptors for biological molecules.
To produce a surface of a catalytically active material.
To produce surfaces with different degrees of wetting properties.

A known method of creating active surfaces is described by Zhang, Yongjun; Yang, Shuguang; Guan, Ying; Miao, Xiaopeng; Cao, Weixiao; Xu, Jian; “Novel alternating polymer adsorption/surface activation self-assembled film based on hydrogen bond.” Thin Solid Films (2003), 437(1,2), 280-284.

WO 2005/121397 discloses a method based on MVD technique for producing films which may form active surfaces. This technique is based on gas phase reactions between two reactants thereby forming a new reactant which reacts with the surface of a substrate.

The method for using ALCVD technique to create hybrid surface layers is described in WO 2006/071126.

The ALCVD technique has now been used for the first time to produce active surfaces.

Accordingly the present invention provides a process for preparation of active surfaces on a substrate by atomic layer gas phase deposition technique characterised in that the process comprises the following steps:
a) —contacting the substrate with a pulse of a precursor with an active part;
b) —reacting the precursor with at least one surface of the substrate;
c) —removing non-reacted precursor and reaction by-products if any;
   where said at least one surface of the substrate prior to the contact in step a) comprises a film comprising at least one organic or inorganic monolayer and where the precursor is respectively inorganic or organic.

In a further aspect the present invention provides a process for preparation of active surfaces comprising the following steps:
a) —contacting the substrate with a pulse of an inorganic precursor;
b) —reacting the inorganic precursor with at least one surface of the substrate;
c) —removing non-reacted inorganic precursor and reaction by-products if any;
d) —contacting the reacted inorganic precursor bound to the surface of said substrate with a pulse of an organic precursor; where the organic precursor is an organic compound with an active part and at least one reactive substituent;
e) —reacting the organic precursor with the inorganic compound bound to the substrate;
f) —optionally, removing non-reacted organic precursor and reaction by-products if any.

In another aspect the present invention provides a process for preparation of active surfaces comprising the following steps:
a) —contacting the substrate with a pulse of an organic precursor;
b) —reacting the organic precursor with at least one surface of the substrate;
c) —removing non-reacted organic precursor and reaction by-products if any;
d) —contacting the reacted organic precursor bound to the surface of said substrate with a pulse of an inorganic precursor; where the inorganic precursor is an inorganic compound with an active part and at least one reactive substituent;
e) —reacting the inorganic precursor with the organic compound bound to the substrate;
f) —optionally, removing non-reacted inorganic precursor and reaction by-products if any.

In on embodiment the inorganic precursor is selected from a group consisting of metal alkyls, metal cycloalkyls, metal aryls, metal amine, metal silylamine, metal halogenides, metal carbonyls and metal chelates, where the metal is selected from the group comprising Al, Si, Ge, Sn, In, Pb, alkali metals, alkaline earth metals, 3d-insertion metals, 4d-insertion metals, 5d-insertion metals, lanthanides and actinides.

In another embodiment the organic precursor is an organic compound with at least two reactive substituents selected from the group comprising —OH, —OR, ═O, —COOH, —SH, —SO₄H, —SO₃H, —PH₂, —PO₄H, —PO₃H, —PRH, —NH₂, —NH₃I, —SeH, —SeO₃H, —SeO₄H, —TeH, —AsH₂, —AsRH, —SiH₃, —SiRH₂, —SiRR′H, —GeH₃, —GeRH₂, —GeRR′H, acid anhydride, amine, alkyl amine, silated amine, halogenated amine, imide, azide and nitroxyl; where R and R′ may be a C₁₋₁₀ aryl, alkyl, cycloalkyl, alkenyl or alkynyl group.

In yet another embodiment the organic precursor is an organic compound with an active part and at least one reactive substituent selected from the group comprising —OH, —OR, ═O, —COOH, —SH, —SO₄H, —SO₃H, —PH₂, —PO₄H, —PO₃H, —PRH, —NH₂, —NH₃I, —SeH, —SeO₃H, —SeO₄H, —TeH, —AsH₂, —AsRH, —SiH₃, —SiRH₂, —SiRR′H, —GeH₃, —GeRH₂, —GeRR′H, acid anhydride, amine, alkyl amine, silated amine, halogenated amine, imide, azide and nitroxyl; where R and R′ may be a C₁₋₁₀ aryl, alkyl, cycloalkyl, alkenyl or alkynyl group.

The selectivity or number of active sites of the desired type produced on the surface may be further enhanced by the use of an electric field during the deposition. The electric field will aid in aligning polar molecules so that the desired functional groups will dominate the termination of the surface.

Further the present invention provides a thin film comprising hybrid monolayers comprising an organic compound and an inorganic compound chemically bound to each other characterized in that the thin film comprises one or more surface layers forming an active surface.

In another aspect the invention provides the use of an active surface produced by the process according to the invention or an active surface according to the invention as surfaces: suitable for adhesion through the use of glue or other means, providing receptors for biological molecules, of catalytically active materials, where upon subsequent types of chemical reactions can take place, with different degrees of wetting properties.

Other preferred embodiments of the present invention are described in the sub claims.

Due to the large number of possible organic and inorganic building units that may be imagined used in constructing such active surfaces, there is an almost unlimited imagined outcome in possible interesting properties that may be produced. Some of these constructions may involve several layers of different metals and organic materials in order to construct the desired properties. The motivation of this work has been to demonstrate that the ALCVD method may extend the different possibilities one has to produce active surfaces.

In the present invention the term “inorganic precursor” is considered to mean any compound comprising an inorganic moiety, which by reaction with a surface of a substrate is bound to the surface, thereby connecting the inorganic moiety to the substrate through a bond or one or more bridging elements and where the inorganic moiety bound to the surface comprises one or more reactive groups for reaction with a different precursor. By the term “inorganic moiety” is meant a moiety that contains at least one metal atom. For instance it may be a compound that is metal organic, organometallic, a halogen compound, carbonyl or any other compound that is able to bring the metal into the gas phase.

In the present invention the term “organic precursor” is considered to mean any compound comprising an organic moiety, which by reaction with a surface of a substrate is bound to the surface, thereby connecting the organic moiety to the substrate through a bond or one or more bridging elements and where the organic moiety bound to the surface comprises one or more reactive groups for reaction with a different precursor.

In the present invention the active part of the organic precursor is the part of the organic or inorganic compound bound to the substrate that forms the active surface.

As used in this context the word “film” covers layers of a material which may form a closed surface or which may have openings.

In the present invention the term “adhesive reactive surface” means any active surface or surface structure with the ability to form a strong joint with another surface through the use of an adhesive.

The active surfaces obtained by the present invention may have applications in almost every type of area possibly imagined. Some of those are:
To produce a surface suitable for adhesion through the use of glue or other means. The active surface provides a connection between the native surface and the means one wish to apply. The termination of the active surface should be tailored to provide the type and arrangement of functional sites suitable for reaction with the glue etc.
To produce a surface providing receptors for biological molecules. By terminating the surface with some special arrangements of functional groups one may mimic receptors for biological molecules or biological material. The particular arrangement of functional groups depends on the biological molecule or biological material in question. These surfaces may be used for many different means, e.g., to selectively bond different organic molecules or biological material, which would be interesting for separation analysis, making the surfaces biocompatible by producing a surface that mimics biological tissue, e.g. the cell membrane (mimic the functional groups in: sphingosine, dihydrosphinosine, phytosphingosine, dehydrophytosphingosine etc.), or that mimics the termination of the bone material (calcium phosphates), etc.
To produce a surface of a catalytically active material. The topmost surface may be viewed upon as a metal organic fragment, and is thus expected to undergo some of the similar types of chemistry found for the similar molecular species. These may be used as heterogeneous catalysis or similar.
To produce a surface where upon subsequent types of chemical reactions can take place. 1) One possible application would be to produce a surface that terminates with groups similar to the monomers forming polymers. It would then be possible to use these surfaces in synthesis and form polymers that are end-bonded to the surfaces and rather well attached. 2) Rather than forming polymers one will have the possibility to construct larger types of molecules that are attached to the said surface. The conversion of the surfaces may be done through “normal” synthesis routes.
To produce surfaces with different degrees of wetting properties. By terminating the surface with selected types of functional groups one will be able to tune the wetting properties of the different surfaces towards different compounds. This may be beneficial in production of anti-fogging surfaces (good wetting properties) or production of surfaces showing the Lotus effect (low wetting properties).

The substrate material is not limited to the type of materials used here but can be any material reactive towards at least on of the precursors.

The surfaces that are activated may take any physical form as long as it is a solid or liquid stable during the deposition conditions. One may use flat substrates, powder or substrates with complex geometries.

### DESCRIPTION OF FIGURES

The present invention will be described in more detail with reference to the following figures:

FIG. 1: Shows a schematic representation of an active surface.

FIGS. 2a and 2b: Illustrates the growth kinetics of trimethyl-aluminium (TMA) and different aromatic acids.

FIG. 3: IR absorbance spectra of thin films of (a) Al-Hq, (b) Al-Phl, (c) Al-Hq-Al-Phl, both as-deposited and air treated. Where Hq stands for hydroquinone and Phl stands for phloroglucinol (1,3,5-trihydroxy-benzen). The films were grown with 1000 cycles at a reactor temperature of 200° C. The absorbance spectra for pure Hq and Phl are added as reference.

FIG. 4: Growth kinetic analysis acquired by quarts crystal monitor (QCM) measurements of (a) Al-Hq, (b) Al-Phl, (c) Al-Malonic acid, TMA, Terephtalic acid, Zr-Hq, Ti-Hq, and (d) Ti-Ethylenediamine growth. The data is based on the average of 20 successive pulses.

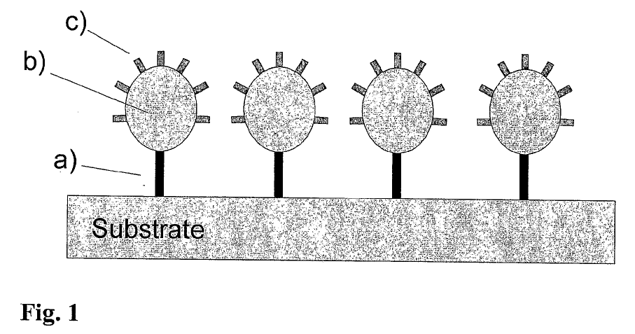

A schematic representation of the terminated surface is shown on FIG. 1. Where a) is a functional group capable of forming bonds to the substrate, b) the organic backbone supporting the functional groups forming the terminated surface (this can be any kind of organic fragment from: linear alkanes, branched alkanes, cyclic compounds, aromatic etc. it may also be a part of the functional groups forming the active surface.) c) are the functional groups terminating the surface and thus producing an active surface.

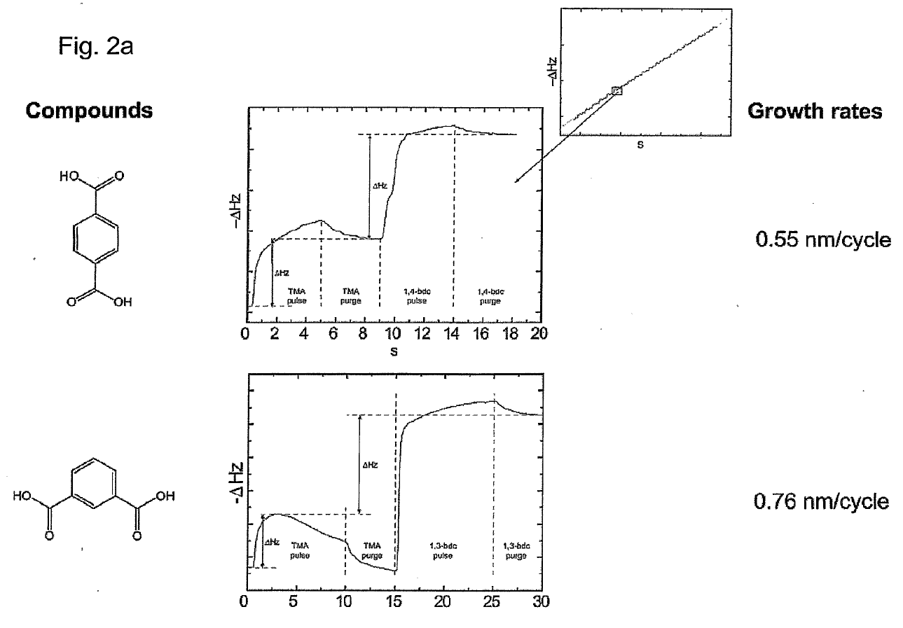

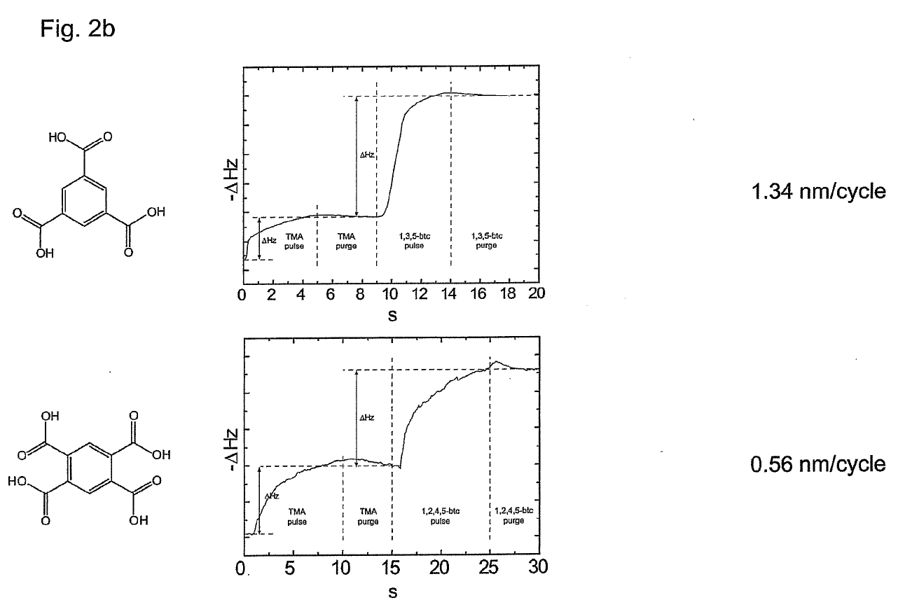

FIGS. 2a and 2b show the growth kinetics of TMA and the aromatic acids 2a: 1,4-benzenedicarboxylic acid, 1,3-benzenedicarboxylic acid, 2b: 1,3,5-benzenetricarboxylic acid, and 1,2,4,5-benzenetetracarboxylic acid.

The ALCVD technique can be viewed as a controlled sequential chemical reaction technique. The film is built by letting a functional group on a gas molecule react with a suitable site on a surface. The gas molecules that react must leave a site on the surface that will function as a reactive site for a following type of gas molecules in order to produce a continuous film. The genuine idea here presented is to end the deposition with a precursor that terminates the surface with the desired type and arrangement of functional groups.

The fact that a control of the surface termination can be achieved is exemplified in the growth of films of organic-inorganic nature as Al-Hq and Al-Phl films. These films grow by that methyl groups attached to aluminium react with hydroxyl groups to form methane and a film of aluminium benzene oxide. The trimethyl-aluminium (TMA) does not react all of its three alkyl groups with the surface due to steric hindrance, but keeps at least one group as a reactive site towards a succeeding gas molecules, The Hq-gas molecule is of a diol type where only one of the hydroxyl groups can react with surface site due to steric hindrance. This will then leave a hydroxyl terminated surface for succeeding reactions with TMA, or rather an active surface of hydroxyl groups attached to an aromatic compound if the reaction is stopped at this point. Alternatively the reaction may be stopped after the TMA pulse, which then will produce a surface terminated by alkyl-groups.

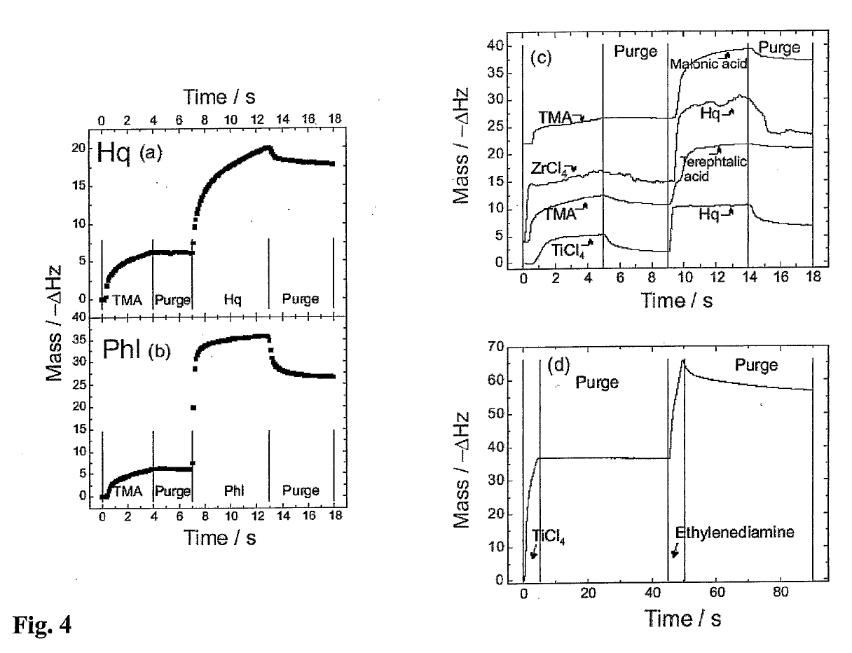

This type of formation of active surfaces has also been exemplified by growth of films with the precursor combinations: TMA and Malonic acid, TMA and Terephtalic acid, ZrCl₄ and Hq, TiCl₄ and Hq, TiCl₄ and Ethylenediamine. The growth kinetics as derived from QCM data from these experiments are shown in FIG. 4. The type of terminating surface is determined by the type of precursor used for the last pulse. FIGS. 2a and 2b show the growth kinetics of TMA and the aromatic acids 2a: 1,4-benzenedicarboxylic acid, 1,3-benzenedicarboxylic acid, and 2b:1,3,5-benzenetricarboxylic acid, and 1,2,4,5-benzenetetracarboxylic acid.

The two/or more types of functional groups on a gas molecule need not be of the same type, but should in any way leave a reactive site suitable for succeeding growth with another type of gas molecules. Each type of gas molecules must not undergo reactions with itself.

For production of the final terminating surface, the requirement of two or more types of functional groups does not apply. Rather the precursor should have at least one functional group that will undergo reactions with the previous surface, and also contain the group(s) that should finally terminate the surface.

Some of the functional groups imagined to undergo reactions by the ALCVD principle and thus suitable as functional groups to attach active molecules to the surface are described in the following. For all the proposed reaction mechanisms, there are high possibilities that the reaction scheme is somewhat shifted or different and the reaction schemes should not be interpreted limiting for the scope of the invention. The main principal is that the reaction results in a film being formed and thus shows the potential for use as functional groups to attach active molecules.

Concerning possible reactions between metal containing precursors and organic molecules with functional groups. Organic molecules with functional groups that have some degree of acidity, wiz, can donate a proton are preferred. This proton will be used to complete the alkane molecule or halogen acid molecule from the inorganic precursor and let the reaction proceed.

Below are presented some functional groups on the organic precursor that can be involved in the reactions, and examples on the reaction are given. All the suggested reactions are only illustrations of possible reactions and are not to be interpreted as limitations.

### Hydroxyl Groups

Hydroxyl groups (R—OH) provide an oxygen and hydrogen for a possible reaction. These react readily with electropositive metals e.g. in the form of alkyls or halogenides, whereby a metal alkoxide and methyl or hydrohalogen acid is produced, respectively. This reaction is demonstrated for the production of Al-Hq and Al-Phl films by TMA and Hq or Phl.

The two partial reactions that take place between a diol (HO—R—OH) and TMA ((CH₃)₃Al) are given below:

HO—R—OH(g)+CH₃—Al—|→CH₄(g)+HO—R—O—Al—|

2HO—R—|+(CH₃)₃Al(g)→2CH₄(g)+CH₃—Al—(O—R—|)₂

Electropositive metals known to readily undergo such reactions are: Al, Mg, Si, Ti, V and most probably several other metals like Zn, Mn, Fe, Co, Cr among others

### Ether Groups

Ether groups (—OR) may react and form adducts to metals in the film. These bonds are rather weak, but still they may be a basis of structure formation for films produced at low temperatures. An example of such a reaction scheme is:

R′O—R(g)+CH₃—Mn—|→R′O—R—Mn—|

2R′—R—|+(CH₃)₃Al(g)→2R′—CH₃(g)+CH₃—Al—(O—R—|)₂

Only one half reaction is presented, because it is rather likely that film formed via this reaction path will use another type of functional group in its structure to form film in the next step. The general idea is to form chelating bonds between the ether and the metal atom.

### Ketone Groups

Ketones (R═O) may react and form chelating bonds to a metal atom. One such example is the formation of compounds with β-ketones. An example of such visualized reaction scheme is:

O═R′—R═O(g)+Mn—|→R,R′(═O)₂—Mn—|

Only one half reaction is presented, because it is rather likely that film formed via this reaction path will use another type of functional group in its structure to form film in the next step. The general idea is to form chelating bonds between the ketone and the metal atom.

### Carboxyl Groups

Carboxyl groups (—COOH) have the same building units as for hydroxyls (—OH) and ketones (═O), and should hence undergo the same reactions that these types show.

The two partial reactions that take place between a dicarboxylic acid (HOOC—R—COOH) and TMA ((CH₃)₃Al) are given below:

HOOC—R—COOH(g)+CH₃—Al—|→CH₄(g)+HOOC—R—COO—Al—|

2HOOC—R—|+(CH₃)₃Al(g)→2CH₄(g)+CH₃—Al—(COO—R—|)₂

There is also a possibility for a different reaction scheme with TMA where it will react with both the ═O and the —OH of a carboxyl acid.

### Thiol Groups

Thiol groups (—SH) should react according to the same pattern as for their isoelectronic hydroxyl relatives (—OH). However, metal affinity towards sulphur is somewhat different than for oxygen. This results in that elements such as Pb, Au, Pt, Ag, Hg, and several more readily will react and form stable bonds towards sulphur.

The two partial reactions that take place between a di-thiol (HS—R—SH) and Pt(thd)₂ are given below:

HS—R—SH(g)+thd-Pt—|→Hthd(g)+HS—R—S—Pt—|

2HS—R—|+Pt(thd)₂(g)→Hthd(g)+thd-Pt—S—R—|

### Sulphate Groups

Sulphate groups (—SO₄H) should react with electropositive metals according to the same reaction paths as for hydroxyls or ketones.

The two partial reactions that take place between a disulphate (HSO₄—R—SO₄H) and TMA ((CH₃)₃Al) are given below:

HSO₄—R—SO₄H(g)+CH₃—Al—|→CH₄(g)+HSO₄—R—SO₄—Al—|

2HSO₄—R—|+(CH₃)₃Al(g)→2CH₄(g)+CH₃—Al—(SO₄—R—|)₂

### Sulphite Groups

Sulphite groups (—SO₃H) should react according to the same pattern as for sulphate groups.

The two partial reactions that take place between a disulphite (HSO₃—R—SO₃H) and TMA ((CH₃)₃ Al) are given below:

HSO₃—R—SO₃H(g)+CH₃—Al—|→CH₄(g)+HSO₃—R—SO₃—Al—|

2HSO₃—R—|+(CH₃)₃Al(g)→2CH₄(g)+CH₃—Al—(SO₃—R—|)₂

### Phosphide Groups

The two partial reactions that take place between a di-phospide (H₂P—R—PH₂) and Ni(thd)₂, where thd stands for 2,2,6,6-tetramethyl-3,5-heptandione, are given below:

H₂P—R—PH₂(g)+thd-Ni—|→Hthd(g)+H₂P—R—PH—Ni—|

2H₂P—R—|+Ni(thd)₂(g)+Hthd(g)+thd-Ni—PH—R—|

### Phosphate Groups

The two partial reactions that take place between a diphosphate (HPO₄—R—PO₄H) and TMA ((CH₃)₃Al) are given below:

HPO₄—R—PO₄H(g)+CH₃—Al—|→CH₄(g)+HPO₄—R—PO₄—Al—|

2HPO₄—R—|+(CH₃)₃Al(g)→2CH₄(g)+CH₃—Al—(PO₄—R—|)₂

### Amine Groups

Amine groups, alkyl amines, or silated amines, or halogenated amines, should react with compounds such as SnI₂, SnI₄, PbI₂, PbI₄, CuI₂, CuI₄ or similar to form perovskite related hybrid materials as described by D. B. Mitzi (D. B. Mitzi, Progress in Inorganic Chemistry, 48 (1999) 1-121. and D. B. Mitzi in Chemistry of Materials 13 (2001) 3283-3298).

One proposed reaction mechanism is:

SnI₄(g)+NH₄—I—R—|→SnI₄—NH₄I—R—|

SnI₄—|+NH₄I—R—H₄NI(g)→NH₄I—R—H₄N—SnI₄—|

Further a redox-reaction with Sn(IV)-Sn(II) and formation of I₂(g) might be involved here. The alternative, by using divalent halogenides may be visualized as:

SnI₂(g)+NH₄I—R—|→SnI₃—NH₄—R—|

SnI₃—|+NH₄I—R—H₄NI(g)→NH₄I—R—H₄N—SnI₄—|

Another reaction mechanism with amines is the analogy of the hydroxides:

The two partial reactions that take place between a diamine (H₂N—R—NH₂) and TMA ((CH₃)₃Al) are given below:

H₂N—R—NH₂(g)+CH₃—Al—|→CH₄(g)+H₂N—R—NH—Al—|

2H₂N—R—|+(CH₃)₃Al(g)→2CH₄(g)+CH₃—Al—(NH—R—|)₂

It is however likely that both H-atoms on one of the amines will react with TMA.

The following functional groups will react in a similar way: —OH, —SH, —SeH, —TeH, —NH₂, —PH₂, —AsH₂, —SiH₃, —GeH₃. —SO₄H, —SO₃H, —PO₄H, —PO₃H, SeO₃H, SeO₄H. In all cases where more than one H is present, the other H may be substituted by another organic group R, where R is straight and branched chain alkane, cycloalkane, an aryl group, a heteroaryl group or one of the other functional groups.

It should be emphasized that both functional groups of a precursor need not be of the same type. By using different functional groups with different reactivity there is a possibility to form a monolayer of organic molecules with a degree of ordering. This could also be combined with usage of different metal components that will have different affinities for the different groups. This ideology could be exploited in order to produce other desired types of terminating surface.

The organic compound carrying the functional groups is not particularly limited but can be any organic molecule that can be brought into the gas phase. It is preferred that there is some steric hindrance in the molecule that will prevent it from reacting with both its reactive groups with the same surface, as there should be some active sites left for the subsequent reaction. Otherwise, there are no limitations to how the organic C—C structure looks. The organic molecule will of course influence the acidity of the protons on the functional groups. The organic compound may be a non-branched alkane, branched alkane, cyclo alkane, alkene, a monocyclic or polycyclic aromatic group, a heterocyclic aromatic group, where these compounds in addition to the functional groups can be substituted or not substituted with other organic groups like alkyl.

The inorganic precursors that can take part in the process according to the invention are described below. All the suggested reactions are only illustrations of possible reactions and are not to be interpreted as limitations.

### Metal Alkyls

Metal alkyls and metal cycloalkyls are rather reactive and hence undergo reaction with most organic functional groups. This is exemplified by production of hybrid thin films by TMA and hydroxides. Examples of possible metal alkyls are: Al(CH₃)₃, Zn(Et)₂, Zn(Me)₂, MgCp₂;

where Cp stands for cyclopentyl.

### Metal Halogenides

Some electropositive metal halogenides are rather reactive and undergo reaction with many organic functional groups. Some examples are AlCl₃, TiCl₄, SiCl₄, SnCl₄, Si(CH₃)₂Cl₂.

### Metal Carbonyls

Metal carbonyls are also reactive, and some examples are: Fe₂(CO)₉, Mn(CO)ₓ

### Metal Chelates

Examples of reactive metal chelates are: VO(thd)₂, Mn(HMDS)₂, Fe(HMDS)₂, TiO(thd)₂, Pt(thd)₂.

Thd (=2,2,6,6-tetramethyl-3,5-heptandione) is a chelating compound from which compounds with elements as Ti, V, and several more can be made. HMDS stands for hexamethyl-disilazane

Other possible chelates are beta-ketones such as acetylacetonates, fluorinated thd-compounds and ethylenediaminetetra acetic acid (EDTA).

The metal for the inorganic precursor is selected from the group consisting of Al, Si, Ge, Sn, In, Pb, alkali metals, alkaline earth metals, 3d-insertion metals, 4d-insertion metals, 5d-insertion metals, lanthanides and actinides. Some of the more interesting metals are Cu, Ni, Co, Fe, Mn and V.

The formation of hybrid thin films is exemplified in the examples below, where trimethylaluminium (TMA), hydroquinone (Hq) and phloroglucinol (Phl) have been used as precursors to fabricate thin films of aluminium benzene oxides constructing a hybrid type of film. Films are produced by usage of TMA-Phl, TMA-Hq, and a controlled mixture of the type TMA-Phl-TMA-Hq. The growth kinetic is investigated on the basis of quarts crystal monitor (QCM) measurements and the films are analysed by Fourier transformed infrared spectroscopy (FT-IR). Active surfaces terminating in hydroxyl groups on aromates or metal alkyls are obtained by using Hq/Phl or TMA respectively as the last type of precursor.

The relative growth rate was measured with a QCM by using the slope of the measured resonant frequency over a 50 growth cycle period and normalizing it to growth per cycle. In this way the growth rate could be expressed in terms of ΔHz cycle⁻¹, which is linearly proportional to a growth rat based on mass cycle⁻¹. The result shows that a self controlled growth can be obtained for properly chosen pulse parameters. The results indicate that the reaction of TMA with Phl terminated film occurs on two types of sites. The first type of sites is saturated at 0.2 s whereas a pulse length of 2 s is necessary in order to saturate the second type fully. According to QCM investigations of the pulse parameters it is possible to grow hybrid films of TMA-Phl and TMA-Hq controlled with the ALCVD technique by using pulse parameters of 1.0 s for Phl and Hq pulse followed in both cases by a purge time of at least 0.5 s, and a pulse period of respectively 1.5, 0.5 s of TMA before a purge of 0.2 s for Phl and Hq containing films. The effect of temperature on the growth rate of TMA-Hq and on TMA-Phl films was measured by x-ray profilometry for films grown with 80 cycles at different reactor temperatures. The thickness measurement was done immediately after deposition and also on the same films after treatment in normal atmosphere for at least one week. This reveals that the films are somewhat air sensitive and alter the thickness with time; however, the thickness alteration is different for the two types of films. Films with Phl grow at a typical growth rate of 0.34 nm/cycle and reduce in thickness by 0.25 nm/cycle by air treatment, whereas films with Hq rather increase in thickness from 0.53 to 0.58 nm/cycle on air treatment. Nevertheless it shows that the growth rate is only minor affected by the growth temperature for both types of film, revealing an ALCVD window of 150-300 and 200-350° C. for Phl and Hq containing films, respectively. The linearity of growth was also tested by QCM measurements over a total of 1000 pulse cycles with the configuration 1.0-1.0-0.9-0.5 s for Hq/Phl pulse-purge-TMA pulse-purge. Both Phl and Hq containing films were found to have a linear thickness dependence on the number of pulse cycles, except for the initial stages of growth (up to ca. 30 pulses) where a reduced growth rate was found. This behaviour may stem from a limited amount of nucleation sites on the gold surface of the quarts resonators, and preferential subsequent growth of the initial nuclei.

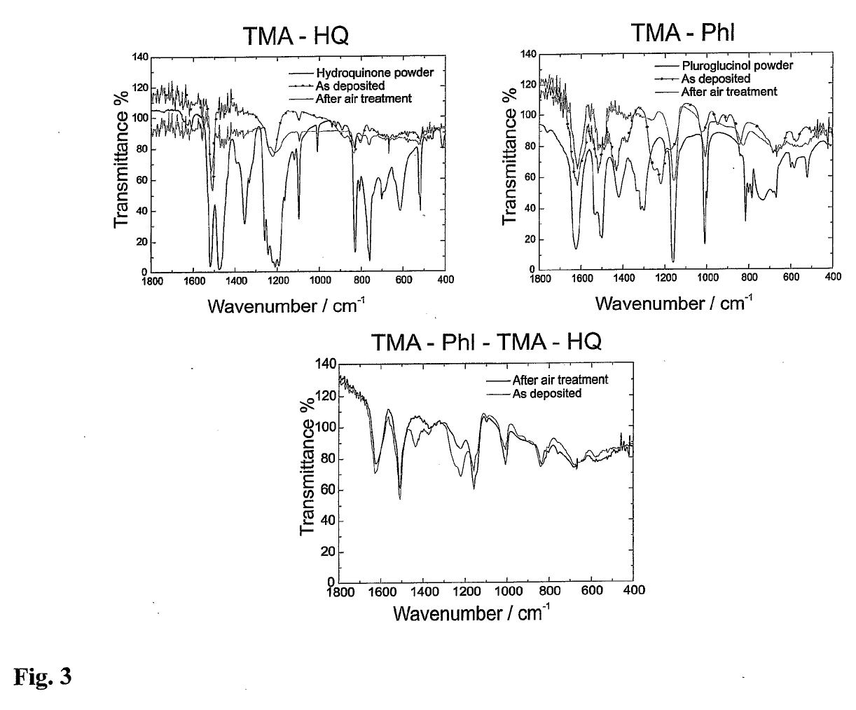

The result of IR analysis of films both as deposited and air treated for at least one week is shown in FIG. 3 together with IR analysis of pure hydroquinone and phloroglucinol pressed in KBr-pellets. This reveals that there are distinct resemblances between the pure constituents and the films formed. There are in addition also some new bands formed that is believed to be due to the formation of the benzene oxides. The IR signals are somewhat altered when the films are air treated revealing that a chemical reaction is taking place. As for the Phl-Hq mixed films, it is possible to identify the pattern from both of the pure types of films. Attempts to measure the growth kinetics by using QCM, was done by measuring the growth pattern over 20 rather long cycles and then arithmetically average the result from all these cycles (FIG. 4). The most intuitively reaction mechanisms for these processes are presented in the equations below for Phl and Hq containing films respectively and give a relatively mass change that depends on the liberated methane stoichiometry for each pulse. The measured relative frequency changes were 3.5 and 1.9 respectively for Phl and Hq containing films. By solving the x parameters with basis in these values, a final methane stoichiometry of x=1.87 and 1.25 equates for Phl and Hq containing films respectively. This may however not be the total truth, since it may just as well be imagined that an excess of Phl and/or Hq may be introduced and leave unreacted HO—| in the film which is sterically hindered from full reaction with TMA. The apparent two sectioned shape of the dependence of the growth rate on the TMA pulse time; may indicate that the last case of sterically hindered HO—| groups, is the most likely case in these situations. XRD analysis of the films proved that neither of the films was well oriented. The as grown films with Phl gave no diffraction pattern whereas the as grown films with Hq had one diffraction peak at 2θ=23.0°. For air treated films a diffraction peak at 2θ=27.5° appeared on films with Phl. For the same type of treated films with Hq, a minor peak at 2θ=18.5° appeared. Rocking curve analysis of peaks on as grown films with Phl and for the peaks on both of the aired films proved that they all were fully randomly oriented. This was also observed for films with both Phl and Hq. The XRD analysis of these films resembles those with only Phl.

IR analysis has been performed on a selection of the exemplified growth systems and a table of the characteristic absorption bands or peaks are given in the table A. It should be taken into consideration that some of these films have undergone reactions with the air while performing the IR measurements.

TABLE A
Characteristic IR absorption bands or peaks for deposited hybrid films.

| Inorganic | Organic | |
|---|---|---|
| precursor | precursor | Absorption bands/cm⁻¹ |
| TMA | Hq | 1507, 1223, 879, 834, 800 |
| TMA | Phl | 1619, 1516, 1433, 1381, 1247, 1218, |
| | | 1173, 1020, 946, 905, 845, 668, 574 |
| TMA | Terephtalic acid | 1596, 1509, 1415, 1312, 1251, 1160, |
| | | 1103, 879, 812, 755, 573 |
| TMA | Malonic acid | 1620, 1485, 1438, 1376, 1280, 1235, |
| | | 1180, 1074, 1003, 965, 812, 738, 522 |
| TiCl₄ | Hq | 1487, 1198, 886, 832, 817 |
| ZrCl₄ | Hq | 1497, 1272, 1203, 884, 832, 804 |

We have shown the possibility of growth of films with a hybrid type by the ALCVD technique by growing films with a network of aluminium benzene oxides with hydroquinone and phloroglucinol. We have made films with aluminium benzene oxides with pure hydroquinone or phloroglucinol and films where these are mixed. Overall we have so fare also shown the possibility of growth of hybrid type films by the ALCVD technique using precursor pairs as:

TMA-Hq, thus proving growth of Al and also growth of an aromatic diol.
TMA-Phl, thus proving growth of also an aromatic triol.
TMA-Malonic acid, thus proving growth with a linear di-carboxcylic acid.
TMA-Terephtalic acid, thus proving growth with an aromatic di-carboxcylic acid.
TiCl₄-Hq, thus proving growth with titanium.
ZrCl₄-Hq, thus proving growth with zirconium.
TiCl₄-Ethylenediamine, thus proving growth of an amine.

### Reaction Equations:

### Principal Reaction Mechanism:

### Hq-Case:

$${Al ( {CH}_{3} )}_{3} ( g ) + 1.5 HO — \vert \_ {( {CH}_{3} )}_{1.5} {Al— O}_{1.5} \text{-} \vert + 1.5 {CH}_{4} ( g )$$

$$Δ ml = 48.02 u \\ 1.5 {C}_{6} {{H}_{4} ( OH )}_{2} ( g ) + {( {CH}_{3} )}_{1.5} {Al — O}_{1.5} — \vert \_ {( {HO — C}_{6} {H}_{4} — O )}_{1.5} {— Al — O}_{1.5} \text{-} \vert + 1.5 {CH}_{4} ( g )$$

$$Δm 2 = 141.10 u$$

$$Δm 2 / Δm 1 = 2.94$$

The mass difference is smaller than measured by QCM and the mechanism should be modified to allow for less unreacted OH groups during the first step.

### Phl-Case:

$${Al ( {CH}_{3} )}_{3} ( g ) + HO — \vert \_ {( {CH}_{3} )}_{2} Al—O \text{-} \vert + {CH}_{4} ( g )$$

$$Δm1 = 56.04 u \\ {C}_{6} {{H}_{3} ( OH )}_{3} ( g ) + {( {CH}_{3} )}_{2} Al — O — \vert {\_HO— C}_{6} {H}_{3} {— ( O )}_{2} — Al — O \vert + 2 {CH}_{4} ( g )$$

$$Δm 2 = 94.02 u$$

$$Δm 2 / Δm1 = 1.68 .$$

The mass difference is smaller than measured by QCM and the mechanism should be modified to allow for less unreacted OH groups during the first step.

Reaction mechanism with varying degree of reactivity of OH-groups:

### Hq-Case:

$${Al ( {CH}_{3} )}_{3} ( g ) + x HO — \vert \_ {( {CH}_{3} )}_{3 - x} {Al — O}_{x} \text{-} \vert + x {CH}_{4} ( g )$$

$$Δm1 = 48.02 u$$

$$3 - x {C}_{6} {{H}_{4} ( OH )}_{2} ( g ) + {( {CH}_{3} )}_{3 - x} {Al — O}_{x} \text{-} \vert \_ {( {HO — C}_{6} {H}_{4} — O )}_{3 - x} {— Al — O}_{x} \text{-} \vert + ( 3 \text{-} x ) {CH}_{4} ( g )$$

$$Δm2 = 141.10 u$$

$$x = 1.87$$

### Phl-Case:

$${Al ( {CH}_{3} )}_{3} ( g ) + x HO — \vert \_ {( {CH}_{3} )}_{3 - x} {Al — O}_{x} \text{-} \vert + x {CH}_{4} ( g )$$

$$Δm 1 = 56.04 u$$

$${C}_{6} {{H}_{3} ( OH )}_{3} ( g ) + {( {CH}_{3} )}_{3 - x} {Al — O}_{x} \text{-} \vert \_x {( {( HO )}_{x} {— C}_{6} {H}_{3} {— ( {O}_{2} )}_{( 3 - x ) / 2} )}_{3 - x} {— Al — O}_{x} \text{-} \vert + ( 3 \text{-} x ) {CH}_{4} ( g )$$

$$Δm 2 = 94.02 u$$

$$x = 1.25$$

Reaction mechanism with fixed degree of reactivity of OH-groups according to observations by QCM:

### Hq-Case:

$${Al ( {CH}_{3} )}_{3} ( g ) + 1.87 HO — \vert \_ {( {CH}_{3} )}_{1.13} {Al — O}_{1.87} \text{-} \vert + 1.87 {CH}_{4} ( g ) \\ Δm 1 = 42.08 u \\ 1.13 {C}_{6} {{H}_{4} ( OH )}_{2} ( g ) + {( {CH}_{3} )}_{1.13} {Al — O}_{1.87} \text{-} \vert \_ {( {HO — C}_{6} {H}_{4} — O )}_{1.13} {— Al — O}_{1.87} \text{-} \vert + 1.13 {CH}_{4} ( g ) \\ Δm 2 = 147.03 u \\ Δm 2 / Δm1 = 3.5$$

### Phl-Case:

$${Al ( {CH}_{3} )}_{3} ( g ) + 1.25 HO — \vert \_ {( {CH}_{3} )}_{1.75} {Al — O}_{1.25} \text{-} \vert + 1.25 {CH}_{4} ( g )$$

$$Δm 1 = 52.03 u$$

$${C}_{6} {{H}_{3} ( OH )}_{3} ( g ) + {( {CH}_{3} )}_{1.75} {Al — O}_{1.25} \text{-} \vert \_ {( HO )}_{1.25} {— C}_{6} {H}_{3} {— ( {O}_{2} )}_{0.875} {— Al — O}_{1.25} \text{-} \vert + 1.75 {CH}_{4} ( g )$$

$$Δm 2 = 98.03 u$$

$$Δm 2 / Δm1 = 1.9$$

The principle function of this invention is a technique/method or procedure to deposit film of an organic-inorganic nature and create an active surface thereon. The film is produced by a gas phase deposition technique exploiting self hindered surface reactions with individual gas phase precursors. The precursors are pulsed sequentially, each followed by a purge with an inert gas, to avoid gas phase reactions. The procedure has previously been used for pure inorganic materials and is called such names as ALCVD (Atomic Layer Chemical Vapour Deposition, or ALE=Atomic Layer Epitaxy, or ALD=Atomic Layer Deposition).

The invention may be used to produce an active surface on different types of organic-inorganic films bound to a substrate, where the film is built by chemical reactions. In one aspect of the present invention the film provides the possibility to bind the active parts to a substrate. In some applications the composition of the hybrid film can be freely selected as long as the first layer of the film binds to the surface of the substrate and the top layer can react with a precursor comprising the desired active part. Hence, all types of materials constructed of organic blocks and inorganic blocks with the ALCVD (ALD, ALE) method or similar methods should be within the scope of this invention. In another aspect of the present invention the physical properties of the film are as such of importance, as the film provides the substrate with valuable properties as well as allowing for the formation of the desired active surface.

Related methods that are mainly sold as CVD methods, but in principle are relatives of ALCVD should also be included. In this aspect all methods where one utilizes the ideology to separate the introduction of different precursors onto the substrates should be included. This may be performed by either moving the substrates between different precursor zones, or moving the precursor lines over the different substrates, or a mixture of both.

The films have been deposited using a F-120 Sat (ASM Mirochemistry) reactor by using trimethylaluminium, hereafter termed TMA, (Crompton, technical quality), hydroquinone (1,4-dihydroxybenzene, hereafter referred to as Hq) (BDH Laboratory Supplies, >99%) and/or phloroglucinol (1,3,5-trihydroxybenzene, hereafter referred to as Phl) (Merck, >99%) as precursors. The temperature of the TMA precursor was held at 20° C. during film growth whereas hydroquinone and phloroglucinol was sublimed at 140° C. and 175° C. respectively.

Nitrogen was produced in house using a Schmidlin Nitrox 3001 generator (99.999% as to N₂+Ar) and used as purging and carrier gas. The pressure of the reactor during growth was maintained at approximately 2 mbar by employing an inert gas flow of 300 cm³ min⁻¹. The pulse and purge parameters of the film growth was investigated by using a quarts crystal monitor (QCM), by using two 5 MHz gold coated crystal sensors and a Matex MT-400 crystal monitor connected to a computer. This constellation made it possible to sample two sensors at the same time with a 10 Hz recording rate.

A Siemens D5000 diffractometer in θ-θ mode, equipped with a göbel mirror producing parallel Cu Kα radiation, was used for x-ray profilometry thickness measurements and for conventional x-ray diffraction analysis.

IR analysis was performed on films deposited on both sides of double polished Si(100) substrates and using a blank Si(100) substrate as reference. A Perkin Elmer FT-IR System 2000 was used for this purpose.

The films were deposited on soda lime and Si(100) substrates by sequentially pulsing of TMA and either hydroquinone or phloroglucinol. Films were also made where the organic pulses was alternately switched between hydroquinone and phloroglucinol in an Al—O-Hq-Al—O-Phl manner.

1-22. (canceled)

23. Process for preparation of active surfaces on a substrate by atomic layer gas phase deposition technique, the process comprising:
a) —contacting the substrate with a pulse of an inorganic precursor;
b) —reacting the inorganic precursor with at least one surface of the substrate;
c) —removing non-reacted inorganic precursor and reaction by-products if any;
d) —contacting the reacted inorganic precursor bound to the surface of said substrate with a pulse of an organic precursor;
e) —reacting the organic precursor with the inorganic compound bound to the substrate to form an organic inorganic hybrid layer;
f) —optionally, removing non-reacted organic precursor and reaction by-products if any;
g) —optionally repeating step a) to f) and with the same or different types of precursors;
h) —repeating step a) to c) and
i) —contacting and reacting the surface obtained after a step h) with a pulse of an organic precursor; where the organic precursor is an organic compound with an active part and at least one reactive substituent
where the active part forms the active surface and where the active surface is
suitable for adhesion, or
provides receptors for biological molecules, or
is catalytic active, or
is applicable for subsequent types of chemical reactions through synthesis, or
has tuned wetting properties.

24. Process for preparation of active surfaces on a substrate by atomic layer gas phase deposition technique, the process comprising:
a) —contacting the substrate with a pulse of an organic precursor;
b) —reacting the organic precursor with at least one surface of the substrate;
c) —removing non-reacted organic precursor and reaction by-products if any;
d) —contacting the reacted organic precursor bound to the surface of said substrate with a pulse of an inorganic precursor;
e) —reacting the inorganic precursor with the organic compound bound to the substrate to form an inorganic organic hybrid layer;
f) —optionally, removing non-reacted inorganic precursor and reaction by-products if any;
g) —optionally repeating step a) to f) and with the same or different types of precursors;
h) —repeating step a) to c) and
contacting and reacting the surface obtained after a step h) with a pulse of an inorganic precursor; where the inorganic precursor is an inorganic compound with an active part and at least one reactive substituent, where the active part forms the active surface and where the active surface is
suitable for adhesion, or
provides receptors for biological molecules, or
is catalytic active, or
is applicable for subsequent types of chemical reactions through synthesis, or
has tuned wetting properties.

25. Process for preparation of active surfaces on a substrate according to claim 23, wherein the inorganic precursor is selected from a group consisting of: metal alkyls, metal cycloalkyls, metal aryls, metal amine, metal silylamine, metal halogenides, metal carbonyls and metal chelates; where the metal is selected from the group consisting of: Al, Si, Ge, Sn, In, Pb, alkali metals, alkaline earth metals, 3d-insertion metals, 4d-insertion metals, 5d-insertion metals, lanthanides and actinides; and the organic precursor with at least two reactive substituents is an organic compound with at least two reactive substituents selected from the group consisting of: —OH, —OR, ═O, —COOH, —SH, —SO₄H, —SO₃H, —PH₂, —PO₄H, —PO₃H, —PRH, —NH₂, —NH₃I, —SeH, —SeO₃H, —SeO₄H, —TeH, —AsH₂, —AsRH, —SiH₃, —SiRH₂, —SiRR′H, —GeH₃, —GeRH₂, —GeRR′H, acid anhydride, amine, alkyl amine, silated amine, halogenated amine, imide, azide and nitroxyl; where R and R′ may be a C₁₋₁₀ aryl, alkyl, cycloalkyl, alkenyl or alkynyl group.

26. Process for preparation of active surfaces on a substrate according to claim 23, wherein, the organic compound with an active part and at least one reactive substituent is an organic compound with an active part and at least one reactive substituent selected from the group consisting of: —OH, —OR, ═O, —COOH, —SH, —SO₄H, —SO₃H, —PH₂, —PO₄H, —PO₃H, —PRH, —NH₂, —NH₃I, —SeH, —SeO₃H, —SeO₄H, —TeH, —AsH₂, —AsRH, —SiH₃, —SiRH₂, —SiRR′H, —GeH₃, —GeRH₂, —GeRR′H, acid anhydride, amine, alkyl amine, silated amine, halogenated amine, imide, azide and nitroxyl; where R and R′ may be a C₁₋₁₀ aryl, alkyl, cycloalkyl, alkenyl or alkynyl group.

27. Process according to claim 23, wherein steps a) to c) are repeated with independently chosen precursors at least once before the steps d) to f) are performed.

28. Process according to claim 23, wherein the process further comprises pretreating the surface with water to saturate the surface with hydroxyl groups.

29. Process according to claim 25, wherein the metal in the inorganic precursor is an electro positive metal and that the organic precursor is an organic compound selected from the group consisting of: straight and branched chain alkanes, cycloalkanes, aryl groups and heteroaryl groups; and the organic compound is substituted with at least two substituents selected from the group consisting of: —OH, —OR, ═O, —COOH, —SH, —SO₄H, —SO₃H, —PH₂, —PO₄H, —PO₃H, —PRH, —NH₂, —NH₃I, —SeH, —SeO₃H, —SeO₄H, —TeH, —AsH₂, —AsRH, —SiH₃, —SiRH₂, —SiRRH, —GeH₃, —GeRH₂, —GeRRH, amine, alkyl amine, silated amine, halogenated amine, imide, azide and nitroxyl.

30. Process according to claim 25, wherein the organic precursor comprises from 2 to 6 substituents selected from the group consisting of: —OH, —OR, ═O, —COOH, —SH, —SO₄H, —SO₃H, —PH₂, —PO₄H, —PO₃H, —PRH, —NH₂, —NH₃I, —SeH, —SeO₃H, —SeO₄H, —TeH, —AsH₂, —AsRH, —SiH₃, —SiRH₂, —SiRRH, —GeH₃, —GeRH₂, —GeRRH, amine, alkyl amine, silated amine, halogenated amine, imide, azide and nitroxyl.

31. Process according to claim 23, wherein the inorganic precursor comprises an electropositive metal, preferably Al, Si, Sn, Zn, Mg, Ti, V, Mn, Fe, Co, Cr or Pt and the organic precursor is an organic compound substituted with 2-6, more preferred 2-3 substituents selected from the group consisting of —OH, —COOH, —SH, —SO₄H, —SO₃H, —PO₄H, —NH₂, and —NH₃I.

32. Thin film comprising hybrid monolayers comprising an organic compound and an inorganic compound chemically bound to each other wherein the thin film comprises one or more surface layers forming an active surface and where the active surface is
suitable for adhesion, or
provides receptors for biological molecules, or
is catalytic active, or
is applicable for subsequent types of chemical reactions through synthesis, or
has tuned wetting properties.

33. Thin film according to claim 32, wherein the active surface comprises the polar part of sphingosine, dihydrosphingosine, phytosphingosine, or dehydrophytosphingosine.

34. Thin film according to claim 32, wherein the active surface comprises phosphates, preferably calcium phosphate.

35. Use of a thin film comprising hybrid monolayers comprising an organic compound and an inorganic compound chemically bound to each other to obtain an active surface on a substrate, where the active surface comprises active organic moieties or active inorganic moieties and the active surface is a surface suitable for adhesion through the use of glue or other adhesive, provides receptors for biological molecules, makes the surface biocompatible, is a catalytically active material, is a surface where upon subsequent types of chemical reactions can take place, or is a surface with different degrees of wetting properties.

36. Substrate comprising a thin film coating produced by the process according to claim 23.

37. Substrate comprising a thin film coating according to claim 32.
